(19) United States
(12) Patent Application Publication
(10) Pub. No.: US 2008/0262488 A1
(43) Pub. Date: Oct. 23, 2008

(54) Tissue treatment system and a method of cosmetic tissue treatment

(75) Inventors: Keith PENNY, Wellesley, MA (US); Nigel M. GOBLE, Newbury (GB)

Correspondence Address: OLIFF & BERRIDGE, PLC, P.O. BOX 320850, ALEXANDRIA, VA, 22320-4850, US

(73) Assignee: RHYTEC LIMITED, Berkshire (GB)

(21) Appl. No.: 12/078,763

(22) Filed: Apr. 4, 2008

Related U.S. Application Data
(60) Provisional application No. 60/907,655, filed on Apr. 12, 2007.

(51) Int. Cl. A61B 18/22; A61B 18/04

(52) U.S. Cl. 606/28; 606/40

(57) ABSTRACT

A tissue treatment system includes a radio frequency (r.f.) generator, a treatment instrument connectible to the generator and to a source of ionisable gas and operable to produce a plasma jet at a nozzle of the instrument when supplied with the ionisable gas and energised by the generator. The generator is adapted to supply treatment energy to the instrument in the form of at least one discrete burst of pulses of r.f. energy, the burst having a preset number n of pulses, where 2≦n≦5.

This application claims the benefit of priority from U.S. Provisional Patent Application No. 60/907,655 filed Apr. 12, 2007.

### FIELD OF THE INVENTION

This invention relates to a tissue treatment system, particularly a skin treatment system which produces a gas plasma jet for application to the surface of skin. The invention also includes a method of skin treatment, particularly a cosmetic method of regenerating the reticular architecture of skin tissue using a gas plasma jet.

### BACKGROUND OF THE INVENTION

A known skin treatment system comprises a radio frequency (r.f.) generator for providing pulses of r.f. energy to a handheld instrument where it is applied to a stream of ionisable gas, such as nitrogen, as a pulsed electric field to produce a plasma jet for application to the surface of the skin. Such a system is disclosed in U.S. Pat. Nos. 6,629,974 and 6,723,091, and U.S. patent application Ser. No. 10/792,765, the entire disclosures of which are incorporated in the present application by reference. As described in these patent specifications, and in U.S. patent application Ser. No. 11/281,594 (the entire disclosure of which is also incorporated herein by reference), application of the plasma jet can be used to regenerate the reticular architecture of the skin tissue adjacent a line or wrinkle in the tissue. Typically, a single plasma pulse is applied to a given area of target tissue. The handpiece is then moved and another pulse applied to treat an adjacent area of tissue, and so on in this manner to cover a complete area of tissue to be treated, successive pulses being applied at neighbouring locations such that a uniform effect is achieved across the complete area. The r.f. generator may be operated to generate a single pulse for each actuation of a user-operated actuator or the pulses may be generated as a series of pulses continuing for as long as the actuator remains depressed, the operator moving the handpiece to select different treatment locations between the generation of consecutive plasma pulses.

As described in U.S. Pat. No. 6,629,974, the known system produces plasma pulses of predetermined energy, typical pulse energy settings being 2, 2.5, 3, 3.5 and 4 joules.

Treatment may be performed in a single “pass” of the instrument over the surface area to be treated, each pass comprising a plurality of plasma pulse applications at successive locations within the area. However, subsequently, a second pass may be applied, sufficient time having elapsed since the first pass to a given area of tissue such that the skin has cooled.

Pulse width and energy settings employed for patient treatment have been determined by pre-clinical study and confirmed by subsequent clinical study as providing beneficial results.

It is an object of the invention to improve the treatment results.

### SUMMARY OF THE INVENTION

According to a first aspect of the invention, a tissue treatment system includes a r.f. generator, a treatment instrument connectible to the generator and to a source of ionisable gas and operable to produce a plasma jet at a nozzle of the instrument when supplied with the ionisable gas and energised by the generator, wherein the generator is adapted to supply treatment energy to the instrument in the form of at least one burst of pulses of r.f. energy, the burst having a preset number n of pulses, where 2≦n≦5. Preferably, each pulse of the burst has a pulse width in the range of from 2 ms to 20 ms, the time interval between each pulse of the burst being less than 100 ms and, more preferably, between 10 ms and 40 ms. Typically, the energy of each pulse of the burst is in the range of from one joule to two joules.

In the case of the pulse burst having at least three pulses, the time interval between successive pairs of pulses may be different.

The preferred system has a user-operated actuator in the form of a footswitch. In one mode of the generator, depression of the footswitch causes the generator to supply a single burst of pulses. In an alternative mode, depression of the footswitch causes the generator to supply a series of bursts of pulses, each burst being as described above. In this case, the pulses are typically produced at a predetermined repetition rate until the actuator is released. Repetition rates of 0.5 Hz to 4 Hz or 5 Hz are preferred, with 2.5 Hz being a typical preferred value. The repetition rate is preferably presettable.

Configuration of the generator may be such that not only is the number of pulses within each burst presettable, but also the time interval between successive pulses and the width and amplitude of the pulses of the burst. Thus, for example, the separation between pulses may be varied between 10 ms and 100 ms and the pulse width may be varied between 2 ms and 20 ms.

In addition, the r.f. power level of the pulses may be preset to different values, typically between 800 W and 2 kW. Although it is possible to supply a stream of ionisable gas to the instrument continuously during application of a series of pulse bursts, it is preferred that the gas is supplied as gas pulses, each pulse commencing in the interval before a respective burst of r.f. pulses and finishing substantially at the same time as the r.f. pulse burst commences or during the respective r.f. pulse burst.

According to another aspect of the invention, a tissue treatment system includes a r.f. generator, a treatment instrument connectable to the generator and to a source of ionisable gas and operable to produce a plasma jet at a nozzle of the instrument when supplied with the ionisable gas an energised by the generator, wherein the system is adapted to supply treatment energy to the instrument in the form of a burst of pulses of r.f. energy, the energy level of the individual pulses of the burst being below the threshold energy level required typically to induce epidermal vacuolation. In the preferred embodiment, the energy of each pulse of the burst is not less than 1 joule but not greater than 2 joules. The energy of the r.f. pulses may be presettable between these two values.

According to a third aspect of the invention, there is provided a method of cosmetically regenerating the reticular architecture of the dermis by the application of thermal plasma energy, wherein the method comprises applying the thermal plasma energy to the skin surface as bursts of plasma pulses, the energy level of the pulses within the burst being below that required typically to induce epidermal vacuolation, and the application of the pulse bursts being substantially uniform over an area of the skin surface to be treated so as to produce a zone of thermal modification in the dermis in which the inflammatory response produces regeneration of the reticular architecture of the dermis. Nitrogen gas may be used as the ionisable gas.

In the preferred method, the thermal plasma energy is applied using a handheld instrument, the instrument being moved between successive treatment locations during the periods between successive pulse bursts. The energy may be applied to a predetermined skin surface area in a plurality of passes, each pass comprising the application of thermal plasma energy at successive treatment locations.

According to a fourth aspect of the invention, there is provided a method of removing photodamaged tissue from an area of the dermis and regenerating the reticular architecture of the dermis in the said area by the application of thermal plasma energy, wherein the method comprises applying the thermal plasma energy to the said skin surface area as a series of bursts of plasma pulses, the energy level of the pulses within each burst being below that required typically to induce epidermal vacuolation, and the application of the pulse bursts being substantially uniform over the said area to produce a zone of thermal damage that includes at least part of the photodamaged papillary dermis, the method further comprising inducing an inflammatory response below the level of thermal damage to produce regeneration of the reticular architecture of the dermis that replaces at least a portion of the photodamaged dermis. Advantageously, the replacement of photodamaged dermis reduces the depth of surface wrinkles, at least part of the solar elastotic changes associated with photodamage are replaced. Typically, the depth of thermal damage removal includes removal of the epidermis and the pigmentary changes associated with the photodamage, as well as pre-malignant cellular changes. Associated with the thermal modification and reticular regeneration, skin laxity and sagging is tightened, at least in part.

According to a further aspect of the invention, a cosmetic method of regenerating the reticular architecture of the skin tissue adjacent to a line or wrinkle in the tissue using a source of thermal energy adapted to supply thermal energy as a plasma pulse burst comprises the step of operating the thermal energy source to form first and second adjacent regions of thermally-modified tissue in the region of the DE Junction associated with said line or wrinkle, said first region overlying said second region and being thermally modified to a greater extent than said second region.

According to yet a further aspect of the invention, there is provided a cosmetic method of regenerating the reticular architecture of the dermis adjacent to a line or wrinkle using a source of thermal energy with a low thermal time constant and adapted to supply thermal energy as a plasma pulse burst, the method comprising the step of operating the thermal energy source and directing it at the surface of the skin adjacent to said line or wrinkle to form first and second adjacent regions of thermally-modified tissue in the region of the epidermis and dermis of the skin, said first region overlying said second region and being thermally modified to an extent that it separates from said second region some days after the delivery of the thermal energy, and the depth of said separation being dependent on the amount of energy delivered and the thermal capacity of the skin.

The invention also includes a method of regenerating the reticular architecture of skin tissue using a plasma jet formed by applying a radio frequency field to a stream of ionisable gas, wherein the method comprises applying the radio frequency field as at least one discrete burst of radio frequency pulses, the burst having a preset number n of pulses, where n is in the range of from 2 to 5.

The applicants have found that applying a plasma jet to the skin surface as a burst of two, three or more individual plasma pulses at a single location, each pulse having an energy amount somewhat lower than the pulse energies typically employed in the known system, the pulses in the burst being separated by a relatively short time such that the skin does not have time to cool significantly between each individual plasma pulse, produces superior neocollagenesis when compared with the same total energy delivery in a single pulse. This improvement is thought to be the result of lower skin surface temperatures at the skin surface but higher temperatures at the dermal-epidermal junction. Theoretical modelling has shown that varying the pulse width, all other factors being equal, alters the temperature profile at a given skin depth over time. Thus, for instance, comparing short and long pulse delivery, application of a given energy amount in a relatively short time results in higher skin surface temperatures, but lower temperatures at significant depths (e.g. at the dermal-epidermal junction), whereas application of the same amount of energy in a relatively long time results in lower skin surface temperature, but higher temperatures at the same depth.

The invention will be described below by way of example with reference to the drawings.

### BRIEF DESCRIPTION OF THE DRAWINGS

In the drawings:

FIG. 1 is a diagrammatic view of a tissue treatment system in accordance with the invention;

FIG. 2 is a longitudinal cross section of a tissue treatment instrument forming part of the system of FIG. 1;

FIG. 3 is a block diagram of a radio frequency generator for use in the system of FIG. 1;

FIGS. 4A, 4B and 4C are oscilloscope plots indicating the operation of a solenoid valve controlling gas flow and the supply of radio frequency energy to the tissue treatment instrument;

FIG. 5 is a histological slide showing reticular regeneration of the reticular architecture of skin tissue obtained following treatment using a known system;

FIG. 6 is a histological slide showing tissue regeneration after treatment using a system in accordance with the invention; and

FIG. 7 constitutes two histological slides from a strip biopsy taken five days after treatment using a system in accordance with the invention.

### DETAILED DESCRIPTION OF THE PREFERRED EMBODIMENT

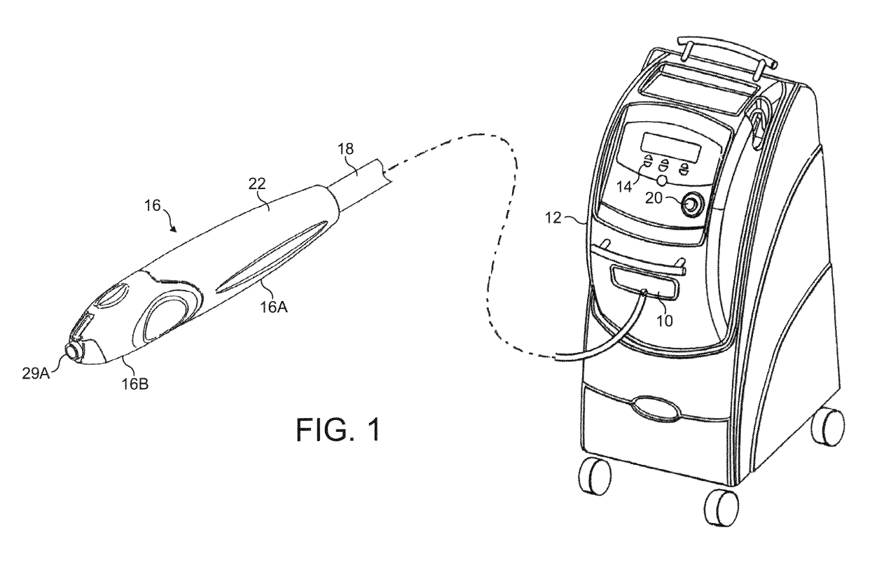

Referring to FIG. 1, a tissue treatment system in accordance with the invention has a treatment power source in the form of an r.f. generator 10 mounted in a floor-standing generator housing 12 and having a user interface 14 for setting the generator to different energy level settings. A handheld tissue treatment instrument 16 is connected to the generator by means of a cord 18. The instrument 16 comprises a handpiece having a re-usable handpiece body 16A and a disposable nose assembly 16B.

The generator housing 12 has an instrument holder 20 for storing the instrument when not in use.

Within the cord 18 there is a coaxial cable for conveying r.f. energy from the generator 10 to the instrument 16, and a gas supply pipe for supplying nitrogen gas from a gas reservoir or source (not shown) inside the generator housing 12. The cord also contains an optical fibre line for transmitting visible light to the instrument from a light source in the generator housing. At its distal end, the cord 18 passes into the casing 22 of the handpiece body 16A

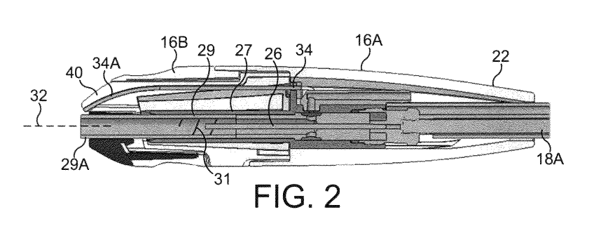

In the re-usable handpiece body 16A, the coaxial cable 18A is connected to inner and outer electrodes 26 and 27, as shown in FIG. 2. The inner electrode 26 extends longitudinally within the outer electrode 27. Between them is a heat-resistant tube 29 (preferably made of quartz) housed in the disposable instrument nose assembly 16B. When the nose assembly 16B is secured to the handpiece body 16A, the interior of the tube 29 is in communication with the gas supply pipe interior, the nose assembly 16B being received within the body 16A such that the inner electrode 26 extends axially into the tube 29 and the outer electrode 27 extends around the outside of the tube 29.

A resonator in the form of a helically wound tungsten coil 31 is located within the quartz tube 29, the coil being positioned such that, when the disposable nose assembly 16B is secured in position on the handpiece body 16A, the proximal end of the coil is adjacent the distal end of the inner electrode 26. The coil is wound such that it is adjacent and in intimate contact with the inner surface of the quartz tube 29.

In use of the instrument, nitrogen gas is fed by a supply pipe to the interior of the tube 29 where it reaches a location adjacent the distal end of the inner electrode 26. When an r.f. voltage is supplied via the coaxial cable to the electrodes 26 and 27, an intense r.f. electric field is created inside the tube 29 in the region of the distal end of the inner electrode. The field strength is aided by the helical coil 31 which is resonant at the operating frequency of the generator and, in this way, conversion of the nitrogen gas into a plasma is promoted, the plasma exiting as a jet at a nozzle 29A of the quartz tube 29. The nozzle 29A has a diameter of 5 mm. The plasma jet, centred on a treatment beam axis 32 (this axis being the axis of the tube 29), is directed onto tissue to be treated, the nozzle 29A typically being held a few millimetres from the surface of the tissue.

The handpiece 16 also contains an optical fibre light guide 34 which extends through the core 18 into the handpiece where its distal end portion 34A is bent inwardly towards the treatment axis defined by the quartz tube 29 to terminate at a distal end which defines an exit aperture adjacent the nozzle 29A. The inclination of the fibre guide at this point defines a projection axis for projecting a target marker onto the tissue surface.

Following repeated use of the instrument, the quartz tube 29 and its resonant coil 31 require replacement. The disposable nose assembly 16B containing these elements is easily attached and detached from the reusable part 16A of the instrument, the interface between the two components 16A, 16B of the instrument providing accurate location of the quartz tube 29 and the coil 31 with respect to the electrodes 26, 27.

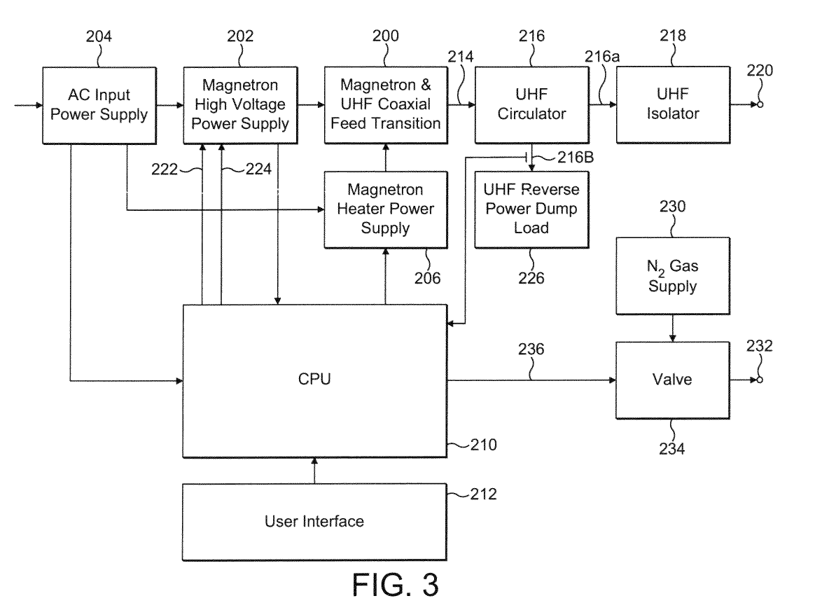

Referring to FIG. 3, r.f: energy is generated in a magnetron 200. Power for the magnetron 200 is supplied in two ways, firstly as a high DC voltage for the cathode, generated by a high voltage power supply 202 supplied from an AC power supply unit 204 and, secondly, as a filament supply for the cathode heater from a heater power supply unit 206. Both the high voltage power supply 202 and the filament power supply 206 are coupled to a CPU controller 210 for controlling the power output of the magnetron. A user interface 212 is coupled to the controller 210 for the purpose of setting the output power mode amongst other functions.

The AC power supply unit 204 is connected to external mains AC power and also generates a supply voltage for the CPU controller 210.

The magnetron 200 and its associated UHF coaxial feed transition generates r.f. energy in the high UHF band, typically at 2.475 GHz, this energy being supplied via a 50 ohm line 214 to a UHF circulator 216 and thence to a UHF isolator 218 constituting a patient isolation barrier. The output 220 from the isolator 218 is connected to the handpiece via a r.f. coaxial cable (neither of which is shown in FIG. 3).

Generation of a high voltage supply output for the magnetron by the high voltage power supply 202 is dependent on two control signals being simultaneously present from the CPU controller 210:

(i) A magnetron current demand signal on line 220 determines the instantaneous r.f. output power level from the magnetron 200 by controlling the high voltage power supply output current fed to the magnetron from the high voltage power supply 202. This output current is proportional to the voltage of the signal on the first control line 222. Since the UHF output power level from the magnetron 200 is proportional to the supply current from the high voltage power supply 202, the magnetron current demand signal on the first control line 222 determines the r.f. output power level from the magnetron.

(ii) An output enable signal on a second control line 224 from the CPU controller 210 turns the high voltage power supply output on and off. The CPU controller 210 governs the output enable control signal to determine the duration of the output current available from the high voltage power supply and, thus, the time during which power is generated by the magnetron 200 and is available on the 50 ohm line 214.

The UHF circulator 216 provides a constant 50 ohm load impedance for the output of the magnetron and its associated UHF coaxial feed transition. Apart from a first port coupled to the magnetron and feed transition stage 200, the circulator 216 has a second port 216A coupled to the UHF isolation stage 218 and a third port 216B which feeds reflected power to a resistive power dump 226. A reflected power sensing connection 228 provides a sensing signal for the controller 210.

Since UHF losses in the UHF circulator 216, the isolator 218, their interconnections (not illustrated) and the coaxial cable feeder to the handpiece (not illustrated) are known or may be otherwise compensated for, the UHF power level at the input to the handpiece can be controlled.

Nitrogen gas for the handpiece is fed through the cord 18 (see FIG. 1) from a pressurised gas supply 230 that is connected to a gas supply outlet 232 coupled to the cord 18. Situated in the gas supply path between the gas supply 230 and the gas supply outlet 232 is a solenoid valve 234 operated by the CPU controller 210 via a control line 236.

When a plasma jet is to be generated, the CPU controller 210 operates to open the solenoid valve 234, allowing gas to pass under pressure to the handpiece, the gas supply control signal being applied to the solenoid valve 234 via the gas supply control line 236. At the same time, the magnetron current demand signal is generated as a voltage level on the first control line 222.

At a predetermined time following opening of the solenoid valve 234, such that gas is flowing in the handpiece (FIG. 1), the CPU controller 210 activates the output enable signal on the second control line 224 so that UHF power is generated at a power level according to the magnitude of the control voltage on the first control line 222. UHF power is generated at a known power level for as long as the output enable signal is present on the second control line 224.

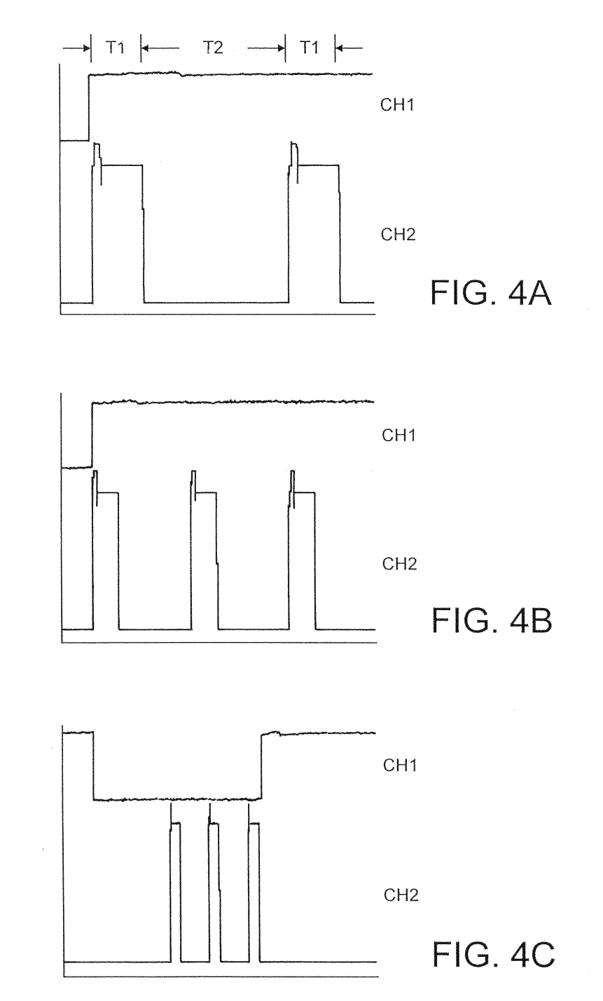

Reference will now also be made to FIG. 4A which shows the gas supply solenoid valve control signal as an upper trace (CH1) and the UHF power output fed from the UHF output terminal 220 as a lower trace (CH2) obtained from an r.f. power detector that monitors the power applied to the handpiece and is displayed as a voltage amplitude. When the gas solenoid valve is closed, no gas flows to the handpiece and the voltage indicated by the upper trace is high. When the valve is open to allow gas to flow, the voltage is low. When r.f. power is supplied from the output terminal 220 (FIG. 3) the lower trace is high, the level being proportional to the output power; when no r.f. power is supplied, the second trace is low.

FIG. 4A illustrates generation of a two-pulse burst of r.f. energy. In this example, the pulse width of each pulse is about 8.2 ms, corresponding to a pulse energy valve of 2 J, and the time interval or separation between the end of the first pulse and the start of the second is 23 ms. As will be seen from the juxtaposition of the upper and lower traces, the gas supply valve is open before the pulse burst is initiated and closes at about the time of pulse burst initiation. This means that during the pulse burst, gas is flowing through the handpiece and can be ionised by the electric field produced in the handpiece by the r.f. pulse burst.

Referring to FIG. 3 in conjunction with FIG. 4A, during generation of a two-pulse pulse burst, as illustrated in FIG. 4A, two pulses of width T1 are generated, each delivering the same amount of energy and separated by a time T2. The CPU controller 210 operates in order that the following actions occur:—

(a) Gas is released by activating the solenoid valve 234 via gas supply control line 236 and then stopped.

(b) The UHF power level is set by a voltage signal on the first control line 222 between the controller 210 and the high voltage power supply 202.

(c) An individual pulse of known power level P1 and pulse width T1 is generated by enabling of the high voltage power supply output via second control line 224 for a period T1 (ignoring propagation and other activation delays that are known and repeatable).

(d) Disabling of the high voltage power supply 202 by removal of the enabling signal on second control line 224 causes cessation of the UHF power output after time T1, and continues for the period T2.

(e) Re-enabling of the high voltage power supply 202 via the second control line 224 for a further period T1 causes resumption of UHF power delivery for the same duration T1 as the first pulse of the burst.

Generation of a three-pulse burst, as shown in FIG. 4B requires repetition of steps (d) and (e). Generation of a four-pulse burst or a five-pulse burst requires repetition of steps (d) and (e) twice or three times respectively.

Settings on the user interface 212 (FIG. 3) determine the pulse burst parameters in terms of the number of pulses in a pulse burst, the energy of individual pulses (being proportional to the r.f. power P1 and pulse duration T1) and the time interval between consecutive pulses. Appropriate timing of gas release is automatically determined by the CPU controller 210 to ensure optimum and consistent plasma generation.

The three-pulse burst illustrated in FIG. 4B comprises three pulses each having a pulse width of about 8 ms and each separated from the neighbouring pulse or pulses by a period of 23 ms. As in the case of the two-pulse burst described above with reference to FIG. 4A, the pulse energy of each pulse is 2 J. The plots shown in FIGS. 4A to 4C represent preferred settings in that pulse bursts having two or three pulses are preferred, the energy delivered in each individual pulse being two joules, yielding a total energy per burst of 4 joules or 6 joules. The total time for the application of each burst is nominally 40 ms for a two-pulse burst and 70 ms for a three-pulse burst. Each burst is preferably applied at a repetition rate of up to 4 Hz.

The two joule per pulse setting is chosen as this approximately corresponds to the maximum energy that does not produce cellular vacuolation that would otherwise provide an insulative effect for a subsequent pulse.

In order that energy delivery by each individual pulse of the burst is known and repeatable, it may be necessary to alter the timing of the gas supply to the handpiece via the solenoid valve 234 (FIG. 3). According to one variation, for instance, the supply of gas may continue throughout the pulse burst, as shown in FIG. 4C. In this case, the solenoid valve is caused to open about 70 ms before commencement of the pulse burst and remains open until the end of the third pulse, the total time during which the solenoid valve 234 is open being about 150 ms in this case.

Generation of the control signals for the magnetron high voltage power supply 202 and the solenoid valve 234 by the CPU controller 210 is under firmware control. Accordingly, firmware settings in conjunction with settings of the user interface determine the r.f. pulse width for each individual pulse within the pulse burst and the timing of the gas solenoid valve activation is such that accurate and predictable energy delivery is achieved in each individual pulse, with a view to optimising the efficiency of plasma generation and minimising gas use. The firmware settings also determine the r.f. power amplitude during each individual pulse. As will be seen from the r.f. power traces of FIGS. 4A to 4C, the r.f. power level during each individual pulse is largely constant except that each pulse has an initial power boost for a brief period following pulse commencement to assist in triggering plasma generation.

In preferential treatments, the instrument 16 is passed over the area of tissue to be treated in one or two passes, the instrument being moved between application of each pulse burst to the skin surface. For a single pass treatment, pulses are preferably applied as sequential lines with the juxtaposition of lines being such as to deliver uniform coverage over the required area. For a two-pass treatment, the sequential lines formed during the first pass are in a first direction and the sequential lines of pulses formed during the second pass are in a second direction with the first and second directions at approximately 90° with respect to each other.

Treatment results obtained with pulse bursts are generally superior to those obtained with single pulses of the prior technique at the maximum pulse width available from the prior system (about 30 ms at 4 joules). Pulse bursts with the same total energy produced improved results.

A two-pass, two-pulse burst, with 2 joules per pulse, 4 joules total, produces improved neocollagenesis compared with the same energy delivered as a single pulse for each of two passes. Zones of thermal damage and modification also seem to be more uniform than those resulting from a single pulse. A two-pass, three-pulse burst produces similar superior results when compared with a single pulse of the same total energy.

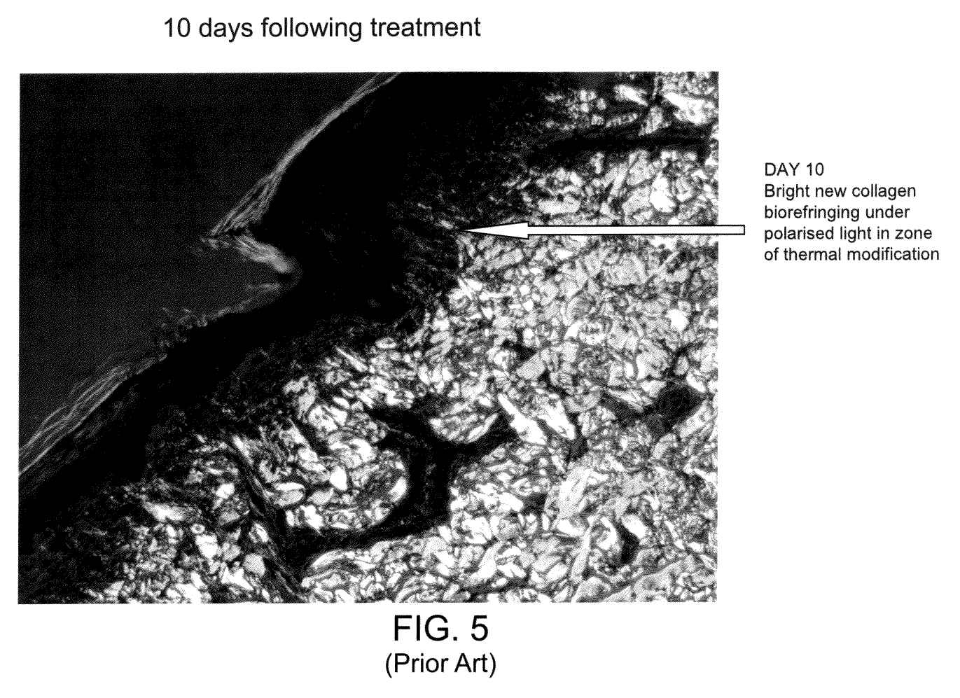

FIG. 5 shows the histology from treatment using the prior system described hereinabove. In this example, a series of single pulses of plasma energy were applied, each having a pulse energy of 3.5 J. The histology shows the skin tissue at 10 days following treatment. The sample is stained with picrosirius red (PSR) so that collagen fibres demonstrate birefringence under polarised light. Some solitary fibres of new collagen are seen lying within the zone of thermal modification which appears dark blue due to the denaturation of the collagen fibres in this zone following treatment.

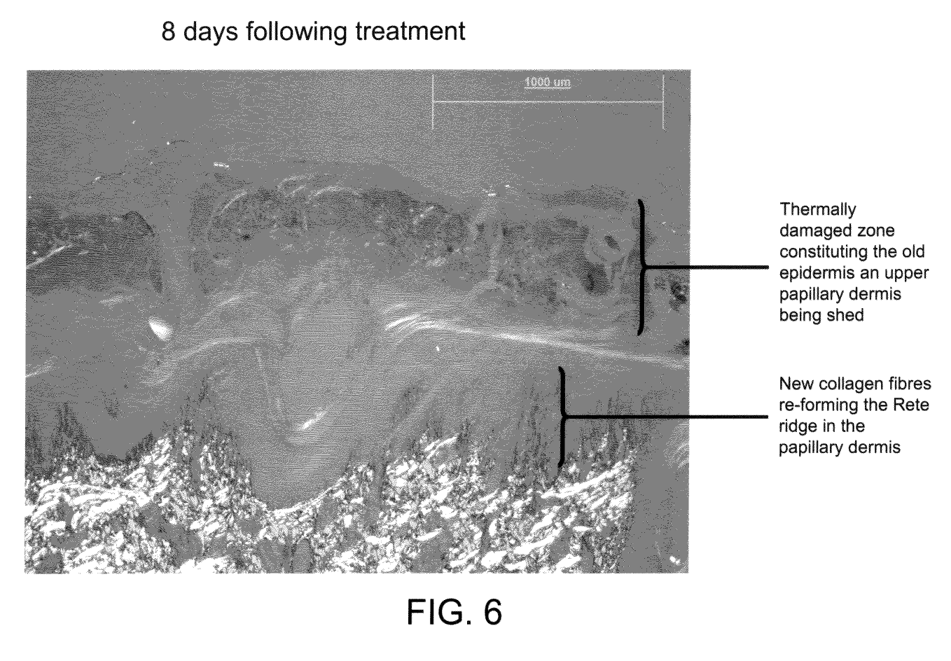

FIG. 6 is a histology of skin tissue at eight days from a double-pass treatment wherein the pulses are applied as discrete pulse bursts in accordance with the invention, using the same method of application, each pulse burst being made up of two pulses of energy and each of such pulses being at an energy level selected to be below the level which induces epidermal vacuolation. The polarisation applied to this slide is somewhat different from that in FIG. 1 such that extensive new collagen fibres laid down in a matrix format are observed forming the new Rete ridges of the dermo-epidermal junction. This represents a far greater level of dermal regeneration of new collagen than has been observed in single pulse treatments.

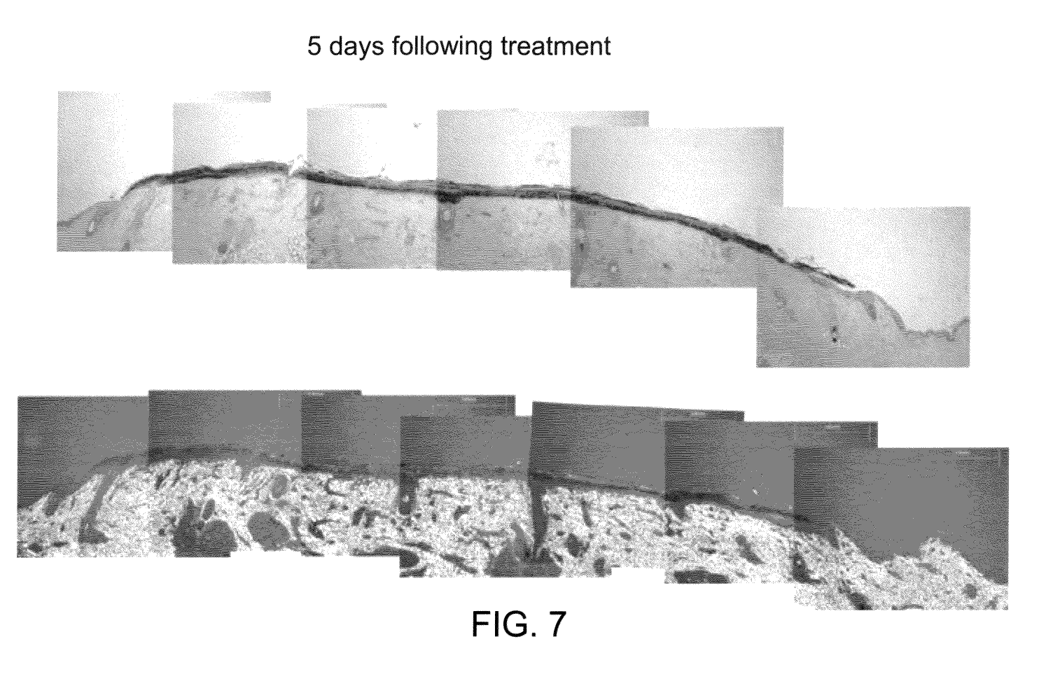

FIG. 7 is the histology from a strip biopsy taken five days following treatment in accordance with the invention, showing the characteristics of the full width of the treated epidermis as a series of sequential electromicrographs stained using H&E (Haemotoxylin & Eosin) and PSR. The notable feature of this histology compared to that obtained with single pulse applications is the greater uniformity of the effect throughout the zone of treatment.

It will be apparent modifications could be made to the system and method described above. For example, the nozzle diameter could lie within the range of from 2 mm to 8 mm. However, the use of the nozzle diameter other than 5 mm would require a scaling of the generator energy setting according to the square of the nozzle diameter.

What is claimed is:

1. A tissue treatment system including a radio frequency (r.f.) generator, a treatment instrument connectible to the generator and to a source of ionisable gas and operable to produce a plasma jet at a nozzle of the instrument when supplied with the ionisable gas and energised by the generator, wherein the generator is adapted to supply treatment energy to the instrument in the form of at least one discrete burst of pulses of r.f. energy, the burst having a preset number n of pulses, where 2≦n≦5.

2. A system according to claim 1, wherein the generator is adapted to supply the burst such that each pulse of the burst has a pulse width in the range of from 2 ms to 20 ms.

3. A system according to claim 1, wherein the generator is adapted to supply the burst such that the time interval between each pulse of the burst is less than 100 ms.

4. A system according to claim 1, wherein the generator is adapted to supply the burst such that the time interval between each pulse of the burst is less than 40 ms.

5. A system according to claim 3, wherein the generator is adapted to supply the burst such that the time interval between each pulse is greater than 10 ms.

6. A system according to claim 1, wherein the generator is adapted to supply the burst such that the energy of each pulse of the burst is in the range of from 1 joule to 2 joules.

7. A system according to claim 1, wherein the generator is adapted to supply the burst such that the burst has at least three pulses and the time interval between respective pairs of pulses is different.

8. A system according to claim 1, having a user-operated actuator, wherein the generator is adapted to operate in a mode in which each operation of the actuator causes the generator to supply a single said burst of pulses

9. A system according to claim 1, having a user-operated actuator, wherein the generator is adapted to operate in a mode in which an operation of the actuator causes the generator to supply a series of said bursts of pulses at a preset burst repetition rate.

10. A system according to claim 9, wherein the generator is adapted such that the burst repetition rate is in the range of from 0.5 Hz to 5 Hz.

11. A system according to claim 1, wherein the generator is arranged such that the time interval between successive pulses of the pulse burst is variable.

12. A system according to claim 1, wherein the generator is arranged such that the width of the pulses of the burst is variable.

13. A tissue treatment system including a radio frequency (r.f.) generator, a treatment instrument connectible to the generator and to a source of ionisable gas and operable to produce a plasma jet at a nozzle of the instrument when supplied with the ionisable gas and energised by the generator, wherein the system is adapted to supply treatment energy to the instrument in the form of a burst of pulses of r.f. energy, the energy level of the individual pulses of the burst being below the threshold energy level required typically to induce epidermal vacuolation.

14. A system according to claim 13, wherein the generator is adapted to supply the burst such that the energy of each pulse thereof is between 1 joule and 2 joules.

15. A system according to claim 13, wherein the generator is adapted to supply the burst such that each burst has at least two but not more than five pulses.

16. A method of cosmetically regenerating the reticular architecture of the dermis by the application of thermal plasma energy, wherein the method comprises applying the thermal plasma energy to the skin surface as discrete bursts of plasma pulses, the energy level of the pulses within the bursts being below that required typically to induce epidermal vacuolation, and the application of the pulse bursts being substantially uniform over an area of the skin surface to be treated so as to produce a zone of thermal modification in the dermis in which the inflammatory response produces regeneration of the reticular architecture of the dermis.

17. A method according to claim 16, wherein the plasma is a plasma of nitrogen gas.

18. A method according to claim 16, wherein the energy level of each pulse is in the range of from 1 joule to 2 joules.

19. A method according to any of claim 16, wherein at least some of the pulse bursts each have at least two but not more than five pulses.

20. A method according to claim 16, wherein the pulses of each of at least some of the bursts each have a pulse width in the range of from 2 ms to 20 ms, the time interval between the pulses of the burst being in the range of from 10 ms to 40 ms.

21. A method according to claim 19, wherein each of at least some of the pulse bursts has at least three pulses and the time intervals between respective pairs of pulses can be varied.

22. A method according to claim 16, wherein the time interval between pulses in each pulse burst is not more than 100 ms.

23. A method according to claim 16, wherein the application of thermal plasma energy to the skin surface is as a series of discrete said plasma pulse bursts, the bursts occurring at a preset repetition rate, the separation between consecutive bursts being between 150 ms to 2 s.

24. A method according to claim 23, wherein the repetition rate is in the range of 30 from 0.5 Hz to 5 Hz.

25. A method according to claim 16, wherein the thermal plasma energy is applied using a handheld instrument, the instrument being moved between successive treatment locations during the periods between successive pulse bursts.

26. A method according to claim 25, in which thermal plasma energy is applied to a predetermined skin surface area in a plurality of passes, each pass comprising the application of thermal plasma energy at successive treatment locations.

27. A method of removing photodamaged tissue from an area of the dermis and regenerating the reticular architecture of the dermis in the said area by the application of thermal plasma energy, wherein the method comprises applying the thermal plasma energy to the said skin surface area as a series of bursts of plasma pulses, the energy level of the pulses within each burst being below that required typically to induce epidermal vacuolation, and the application of the pulse bursts being substantially uniform over the said area to produce a zone of thermal damage that includes at least part of the photodamaged papillary dermis, the method further comprising inducing an inflammatory response below the level of thermal damage to produce regeneration of the reticular architecture of the dermis that replaces at least a portion of the photodamaged dermis.

28. A method according to claim 27, wherein the replacement of photodamaged dermis reduces the depth of surface wrinkles.

29. A method according to claim 27, wherein the replacement of the photodamaged dermis eliminates and replaces at least part of the solar elastotic changes associated with photodamage.

30. A method according to claim 27, wherein the depth of thermal damage removes the epidermis and the pigmentary changes associated with photodamage.

31. A method according to claim 27, wherein the depth of thermal damage removes the epidermis and the pre-malignant cellular changes associated with photodamage.

32. A method according to claim 27, wherein the thermal modification and reticular regeneration tighten at least in part skin laxity and sagging.

33. A cosmetic method of regenerating the reticular architecture of skin tissue adjacent to a line or wrinkle in said tissue, using a source of thermal energy adapted to supply thermal energy as a plasma pulse burst, the method comprising the step of operating the thermal energy source to form first and second adjacent regions of thermally-modified tissue in the region of the DE Junction associated with said line or wrinkle, said first region overlying said second region and being thermally modified to a greater extent than said second region.

34. A cosmetic method of regenerating the reticular architecture of the dermis adjacent to a line or wrinkle using a source of thermal energy with a low thermal time constant and adapted to supply thermal energy as a plasma pulse burst, the method comprising the step of operating the thermal energy source and directing it at the surface of the skin adjacent to said line or wrinkle to form first and second adjacent regions of thermally-modified tissue in the region of the epidermis and dermis of the skin, said first region overlying said second region and being thermally modified to an extent that it separates from said second region some days after the delivery of the thermal energy, and the depth of said separation being dependent on the amount of energy delivered and the thermal capacity of the skin.

35. A method of regenerating the reticular architecture of skin tissue using a plasma jet formed by applying a radio frequency field to a stream of ionisable gas, wherein the method comprises applying the radio frequency field as at least one burst of radio frequency pulses, the burst having a preset number n of pulses, where n is in the range of from 2 to 5.

36. A method according to claim 35, wherein a single burst of pulses is generated upon each operation of a user-operated actuator.

37. A method according to claim 35, wherein a series of discrete bursts of pulses is generated upon operation of a user-operated actuator, such bursts being produced until the actuator is released.

38. A cosmetic method of regenerating the reticular architecture of skin tissue that has degenerated to form a line or wrinkle in said tissue, using a source of thermal energy adapted to supply thermal energy as a plasma pulse burst, the method comprising the step of operating the thermal energy source to form first and second adjacent regions of thermally-modified tissue in the region of the DE Junction associated with said line or wrinkle, said first region overlying said second region and being thermally modified to a greater extent than said second region.

39. A cosmetic method of regenerating the reticular architecture of the dermis that has degenerated to form a line or wrinkle using a source of thermal energy with a low thermal time constant, the method comprising the step of operating the thermal energy source to produce a plasma pulse burst and directing it at the surface of the skin to be treated to form first and second adjacent regions of thermally-modified tissue in the region of the epidermis and dermis of the skin, said first region overlying said second region and being thermally modified to an extent that it separates from said second region some days after the delivery of the thermal energy, and the depth of said separation being dependent on the amount of energy delivered and the thermal capacity of the skin.
